(19) United States
(12) Patent Application Publication
(10) Pub. No.: US 2024/0398353 A1
(43) Pub. Date: Dec. 5, 2024

(54) AMBIENT TEMPERATURE MONITOR

(71) Applicant: NEC Corporation Of America, Herzlia (IL)

(72) Inventor: Tsvi LEV, Tel-Aviv (IL)

(73) Assignee: NEC Corporation Of America, Herzlia (IL)

(21) Appl. No.: 18/203,081

(22) Filed: May 30, 2023

(51) Int. Cl. A61B 5/00; A61B 5/01; G16H 50/20

(52) U.S. Cl. CPC A61B 5/7275; A61B 5/01; G16H 50/20

(57) ABSTRACT

There is provided a system for monitoring temperature changes associated with risk of a medical condition of a subject, comprising: at least one processor in communication with a temperature sensor configured for sensing an ambient temperature and executing a code for: computing a trend of a plurality of temperature measurements of the ambient temperature obtained over a time interval by the temperature sensor, wherein the trend includes differentials between successive temperature measurements and preceding temperature measurements, and analyzing the trend including the differentials to determine risk of the medical condition of the subject.

### FIELD AND BACKGROUND OF THE INVENTION

The present invention, in some embodiments thereof, relates to signal processing for analyzing temperature measurements and, more specifically, but not exclusively, to analyzing temperature measurements for reducing risk of illness.

When temperature falls, a change in temperature from hot to cold can be harmful to the human body, especially for people who have chronic cold-related problems.

### SUMMARY OF THE INVENTION

According to a first aspect, a system for monitoring temperature changes associated with risk of a medical condition of a subject, comprises: at least one processor in communication with a temperature sensor configured for sensing an ambient temperature and executing a code for: computing a trend of a plurality of temperature measurements of the ambient temperature obtained over a time interval by the temperature sensor, wherein the trend includes differentials between successive temperature measurements and preceding temperature measurements, and analyzing the trend including the differentials to determine risk of the medical condition of the subject.

According to a second aspect, a computer implemented method for monitoring temperature changes associated with risk of a medical condition of a subject, comprises: computing a trend of a plurality of temperature measurements of the ambient temperature obtained over a time interval by a temperature sensor, wherein the trend includes differentials between successive temperature measurements and preceding temperature measurements, and analyzing the trend including the differentials to determine risk of the medical condition of the subject.

According to a third aspect, a non-transitory medium storing program instructions for monitoring temperature changes associated with risk of a medical condition of a subject, which when executed by at least one processor, cause the at least one processor to: compute a trend of a plurality of temperature measurements of the ambient temperature obtained over a time interval by a temperature sensor, wherein the trend includes differentials between successive temperature measurements and preceding temperature measurements, and analyze the trend including the differentials to determine risk of the medical condition of the subject.

In a further implementation form of the first, second, and third aspects, further comprising analyzing a plurality of location measurements obtained over the time interval by a location sensor associated with the temperature sensor, to identify whether the trend occurred while the temperature sensor was moved independently of the subject, and in response to identifying that the temperature sensor was moved independently of the subject, excluding the trend from the analysis to determine risk.

In a further implementation form of the first, second, and third aspects, computing the trend comprises computing a spatiotemporal trend of a plurality of the temperature measurements assigned the plurality of location measurement.

In a further implementation form of the first, second, and third aspects, identifying whether the trend occurred while the temperature sensor was moved independently of the subject comprises identifying at least one of: acceleration and/or change in pose of the location sensor, correlated with a local movement of the temperature sensor from a first position on the subject to a second position on the subject.

In a further implementation form of the first, second, and third aspects, further comprising analyzing a plurality of location measurements obtained over the time interval by a location sensor associated with the temperature sensor, and further comprising code for analyzing the plurality of location measurements for identifying that the trend occurred while the temperature sensor was moved as a result of movement the subject, and in response to identifying that the temperature sensor was moved as the result of movement of the subject, analyzing the trend to determine risk.

In a further implementation form of the first, second, and third aspects, identifying that the trend occurred while the temperature sensor was moved as a result of movement the subject comprises identifying a change in location greater than a threshold indicating movement of the subject from a first environment to a second environment.

In a further implementation form of the first, second, and third aspects, the analyzing the trend comprises identifying a rate of the decrease faster than a threshold.

In a further implementation form of the first, second, and third aspects, the medical condition comprises a respiratory viral infection.

In a further implementation form of the first, second, and third aspects, further comprising code for generating instructions for treating the subject for preventing the risk of the respiratory viral infection by generating instructions for reducing the risk by at least one of: moving to a different environment with a warmer ambient temperature, wearing or re-wearing warmer garments, and activating a heater for increasing the ambient temperature.

In a further implementation form of the first, second, and third aspects, further comprising code for computing a second trend of a plurality of physiological measurements of the subject obtained over the time interval by at least one physiological sensor, wherein analyzing comprises analyzing the trend of the plurality of temperature measurements in combination with the second trend of the plurality of physiological measurements.

In a further implementation form of the first, second, and third aspects, the analyzing the trend comprises identifying a rate of increase in temperature greater than a threshold.

In a further implementation form of the first, second, and third aspects, further comprising code for computing a second trend of a plurality of humidity measurements obtained over the time interval by at least one humidity sensor, wherein analyzing comprises analyzing the trend of the plurality of temperature measurements in combination with the second trend of the plurality of humidity measurements.

In a further implementation form of the first, second, and third aspects, further comprising a plurality of body temperature sensors configured to sense body temperature of the subject, the plurality of body temperature sensors positioned at a plurality of spaced-apart locations, each body temperature sensor sensing a respective plurality of body temperature measurements over the time interval, wherein computing the trend further comprises computing a spatiotemporal trend of the body temperature over the time interval from the plurality of temperature sensors, and wherein analyzing further comprises analyzing the spatiotemporal trend.

In a further implementation form of the first, second, and third aspects, analyzing the trend comprises feeding the plurality of temperature measurements into a machine learning model, and obtaining an indication of the risk as an outcome of the machine learning model, wherein the machine learning model is trained on a multi-record training dataset, wherein a record includes a trend of a plurality of sample temperature measurements made by the temperature sensor associated with a sample individual, and a ground truth indication of whether the sample individual developed the medical condition.

In a further implementation form of the first, second, and third aspects, further comprising at least one body temperature sensor configured for sensing a body temperature of the subject, the at least one body temperature sensor in communication with the at least one processor, wherein the trend comprises a first trend, and code for computing a second trend of a plurality of body temperature measurements of a body of the subject over the time interval by the at least one body temperature sensor, wherein analyzing comprise analyzing a combination of the first trend and the second trend to determine risk of the medical condition of the subject.

In a further implementation form of the first, second, and third aspects, the at least one body temperature sensor is installed within a wearable garment, selected from: (i) a hat, and the at least one body temperature sensor is positioned for sensing temperature of a scalp of the subject when in use, (ii) a scarf, and the at least one body temperature sensor is positioned for sensing temperature of air exhaled from nostrils and/or mouth of the subject when in use, (iii) earmuffs, and the at least one body temperature sensor is positioned for sensing temperature of at least one car of the subject when in use, (iv) a shirt, and the at least one body temperature sensor is positioned for sensing temperature of at least one armpit of the subject when in use, and (v) a shirt and/or pants, and the at least one body temperature sensor is positioned for sensing temperature of at least one arm and/or leg and/or a chest of the subject when in use.

In a further implementation form of the first, second, and third aspects, further comprising code for generating instructions for reducing the risk, including at least one of: (i) generating instructions for activating an air conditioner and/or air heater until subsequent trends over subsequent time intervals indicate a substantially stable temperature, (ii) activating a lock on a button and/or a zipper of a wearable garment of the subject for preventing removal of the wearable garment until subsequent trends over subsequent time intervals indicate a substantially stable temperature, (iii) generating an alert indicating the risk, (iv) generating at least one of an audio message played over speakers and a visual presentation on a display indicating to move back to the previous environment and/or add additional clothing and/or turn on a heater or air conditioner.

In a further implementation form of the first, second, and third aspects, the time interval comprises a current time interval, and analyzing the trend comprises analyzing the trend based on the plurality of temperature measurements obtained over the current time interval in combination with historical trends of historical temperature measurements computed over preceding time intervals prior to the current time interval.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic of a block diagram of components of a system for computing and analyzing a trend that includes differentials between temperature measurements, in accordance with some embodiments of the present invention; and

FIG. 2 is a flowchart of a method for computing and analyzing a trend that includes differentials between temperature measurements, in accordance with some embodiments of the present invention.

### DESCRIPTION OF SPECIFIC EMBODIMENTS OF THE INVENTION

The present invention, in some embodiments thereof, relates to signal processing for analyzing temperature measurements and, more specifically, but not exclusively, to analyzing temperature measurements for reducing risk of illness.

As used herein, the term risk refers to risk of developing a new medical condition, onset of a known medical condition which may be dormant and/or of a previous medical condition, and/or exacerbation of an existing active medical condition. Exemplary medical conditions are described herein.

An aspect of some embodiments of the present invention relates to systems, methods, computing devices, and/or code instructions (stored on a data storage device and executable by one or more processors) for monitoring temperature changes associated with risk of a medical condition of a subject, and/or to provide a general wellbeing and/or comfort to the subject from temperature changes. A processor is in communication with at least one ambient temperature sensor that senses an ambient temperature. The temperature sensor generates multiple measurements of the ambient temperature over a time interval. The processor computes a trend of the ambient temperature measurements obtained over the time interval. The trend includes differentials between successive temperature measurements and preceding temperature measurements, for example, detecting a sudden significant drop in temperature over a short time interval, for example, due to a subject that is in a heated home stepping outside to a porch in winter. The trend may include computing a rate of the temperature change, i.e., the change in degrees over an amount of time, for example, decrease in temperature per second. The trend including the differentials may be analyzed. The analysis may be for determining risk of the medical condition of the subject, for example, whether the trend is associated with risk of infection with a respiratory virus (e.g., common cold), risk of exacerbation of an existing medical condition, and/or risk of recurrence and/or onset of a previous medical condition, and the like. Alternatively or additionally, the trend may be analyzed to determine whether the subject is expected to be uncomfortable from the temperature change, for example, a sudden drop in temperature may be uncomfortable, whereas a slower change in temperature may be unnoticeable by the subject. The subject may be treated accordingly, such as to avoid and/or reduce risk of the medical condition, for example, by adjusting the ambient temperature and/or moving locations to where the ambient temperature is not associated with risk.

At least some implementations of the systems, methods, computing devices, and/or code instructions (stored on a data storage device and executable by one or more processors) described herein address the technical problem of detecting rapid changes in ambient temperature which may lead to developing a medical condition, for example, infection with a respiratory virus (e.g., common cold), migraine, exacerbation of sinusitis, cardiovascular issues, exacerbation of arthritis, dryness of skin, worsening of asthma and/or bronchitis, overheating. and the like. The rapid changes may be, from a hot environment to a cold environment, and/or from a cold environment to a hot environment. For example, by a person stepping out from a warm home to a cold outdoor porch. In another example, by a person taking off a warm jacket when the ambient temperature is low.

At least some implementations of the systems, methods, computing devices, and/or code instructions described herein improve the technical field of monitor subjects, and/or reducing risk of medical conditions, and/or signal processing.

At least some implementations of the systems, methods, computing devices, and/or code instructions described herein improve upon prior approaches. For example, some prior approaches monitor the body temperature of a subject to determine whether the body temperature rises above a maximum value or decreases below a minimum value. In another example, another device is designed to monitor and detect hypothermia, by determining if one or more parameters are outside an acceptable range.

At least some implementations of the systems, methods, computing devices, and/or code instructions described herein address the aforementioned technical problem, and/or improve upon the aforementioned technical field, and/or improve upon the aforementioned prior approaches, by analyzing trends of changes of temperature over time, for example, the rate of the change of temperature, whether the change of temperature is due to location change of a subject or location change of the temperature sensors (e.g., removed from an inner pocket), and the like. The approaches described herein enable determining scenarios which may be correlated with high risk of developing a medical condition, enabling the subject to take action to prevent or reduce the risk, for example, return to a warm environment, put on a warm garment, and/or turn on a heater.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

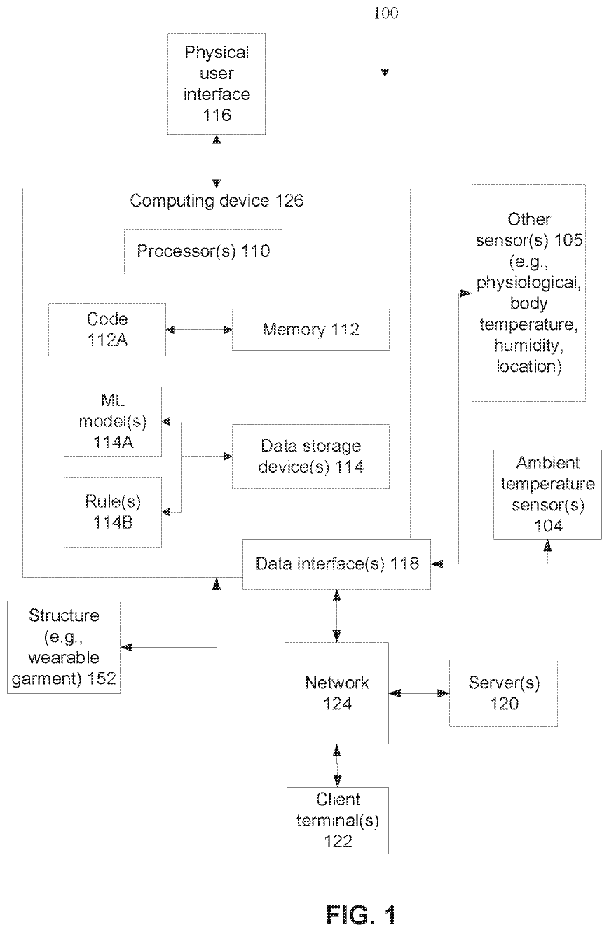

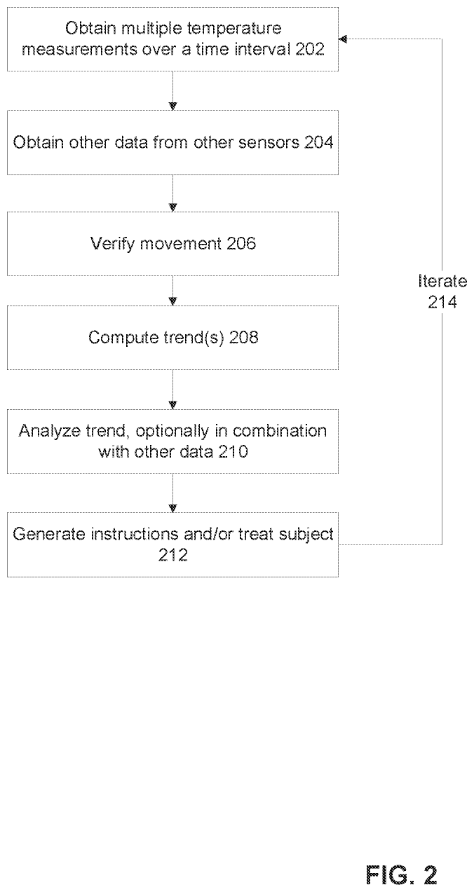

Reference is now made to FIG. 1, which is a schematic of a block diagram of components of a system 100 for computing and analyzing a trend that includes differentials between temperature measurements, in accordance with some embodiments of the present invention. Reference is also made to FIG. 2, which is a flowchart of a method for computing and analyzing a trend that includes differentials between temperature measurements, in accordance with some embodiments of the present invention.

System 100 may implement the acts of the method described with reference to FIG. 2, by processor(s) 110 of a computing device 126 executing code instructions (e.g., code 112A) stored on a memory 112.

Processor(s) 110 of computing device 126 compute a trend of ambient temperature measurements over a time interval made by one or more ambient temperature sensors(s) 104, for example, based on rule(s) 114B and/or feeding the temperature measurements into a machine learning model(s) 114A, and/or other approaches. The ambient temperature measurements are of an ambient temperature in proximity to a subject. Processor(s) 110 may analyze additional data in combination with the temperature measurements, for example, one or more physiological parameter(s) of the subject sensed by one or more physiological sensor(s), and/or location of the temperature sensor(s) 104 made by one or more location sensor(s), and/or other data collected by other sensors 105 as described herein. Processor(s) 110 may generate instructions, for example, an alert, presentation on a display, activation of an air conditioner, and the like, as described herein.

The trend may be computed based on differentials of subsequent temperature measurements and preceding temperature measurements, for example, computing a rate of a drop in temperature, for example, as described herein.

Optionally, there is a single ambient temperature sensor 104 that senses ambient temperature over a time interval. The single ambient temperature sensor 104 enables computing differentials of the ambient temperature at the location where the ambient temperature sensor 104 is located for each temperature measurement. This is in contrast, for example, to using multiple ambient temperature sensors at different locations, for example, a sensor indoors and a sensor outdoors.

Optionally, processor(s) 110 and/or memory 112 and/or data storage device 114 (e.g., of computing device 126), temperature sensor(s) 104, and optionally one or more other sensor(s) 105 are installed within and/or on a structure 152, for example, in a housing (e.g., wristband, pendant on a necklace, small box such as size of a box of matches), within a wearable garment, and the like. In some embodiments, computing device 126 is implemented as a mobile device, for example, a smartphone.

Examples of wearable garment 152 include one or more of: hat, scarf, undershirt, button down shirt, sweatshirt, coat, pants, underwear, socks, and the like. Temperature sensor(s) 104 may be positioned within wearable garment 152 for monitoring body temperature at selected anatomical sites when worn (i.e., in use), as described herein. Alternatively or additionally, temperature sensor(s) 104 may be positioned on structure 152 for monitoring ambient temperature, as described herein.

Wearable garment 152 may be, for example, for covering a head, a chest, a torso, one or more limbs, and the like. Wearable garment 152 may exclude a bracelet. Alternatively, wearable garment 152 may include a bracelet.

Optionally, other sensor(s) 105 include one or more body temperature sensors that sense a body temperature of a subject. The body temperature sensor may be designed to measure body temperature by contact with the body, and/or by a non-contact approach.

Optionally, the other sensor(s) 105, such as the body temperature sensor(s) and/or other sensors such as heart rate sensors, are installed within the wearable garment 152, for example, integrated within the wearable garment 152. Exemplary wearable garments, and installation of one or more physiological sensor(s) 105, such as body temperature sensors, include:
A hat. The other (e.g., body temperature) sensor(s) 105 may be positioned for sensing temperature of a scalp of the subject when in use (i.e., when the hat is worn and the body temperature sensor is in communication with the processor).
A scarf. The other (e.g., body temperature) sensor(s) 105 may be positioned for sensing temperature of air exhaled from nostrils and/or mouth of the subject when in use.
Earmuffs. The other (e.g., body temperature) sensor(s) 105 may be positioned for sensing temperature of one or both ears of the subject when in use.
A shirt. The other (e.g., body temperature) sensor(s) 105 may be positioned for sensing temperature of one or body armpits of the subject when in use.
A shirt and/or pants. The other (e.g., body temperature) sensor(s) 105 may be positioned for sensing temperature of arm(s) and/or leg (s_ and/or a chest of the subject when in use.

Temperature sensor(s) 104 sense temperature of the ambient environment, of the body of the subject, and/or within the wearable garment. Temperature sensor(s) 104 may be contact (e.g., to sense temperature of the body) and/or non-contact. Examples of temperature sensor(s) 104 include: infrared (IR), near infrared (NIR), short wave infrared (SWIR), integrated circuit (IC) temperature sensors, thermocouples, and resistance temperature detectors.

Optional sensor(s) 105 may include physiological sensors(s) that sense one or more physiological parameters of the subject. Examples of physiological sensor(s) include: heart rate sensor for sensing a heart rate of the subject, pulse oximeter sensor for sensing oxygen saturation, electromyogram (EMG) sensor for sensing electrical activity of tissues, and the like.

Other sensor(s) 105 may include, for example, location sensors, body temperature sensors, humidity sensors, and/or physiological sensors. As used herein, the term location sensor(s) refers to a sensor that senses location, and/or acceleration, and/or pose, and/or motion, including a geographical location sensor, a motion sensor, an accelerometer, a position sensor, an inertial measurement unit (IMU), and the like.

Computing device 126 may be implemented as for example, one or more and/or combination of: a standalone component (e.g., within a housing) in communication with structure 152, a group of connected devices, a client terminal, a server, a computing cloud, a virtual server, a computing cloud, a virtual machine, a desktop computer, a thin client, a network node, a network server, and/or a mobile device (e.g., a Smartphone, a Tablet computer, a laptop computer, a wearable computer, glasses computer, and a watch computer).

Computing device 126 includes one or more processor(s) 110, which may interface with sensor(s) 104-105. Processor(s) 110 may interface with other components, described herein. Processor(s) 110 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 110 may include a single processor, or multiple processors (homogenous or heterogeneous) arranged for parallel processing, as clusters and/or as one or more multi core processing devices.

Computing device 126 includes a memory 112, which stores code 112A for execution by processor(s) 110. Code 112A may include program instructions for implementing one or more features of the method described with reference to FIG. 2, as described herein. Memory 112 may be implemented as, for example, a random access memory (RAM), read-only memory (ROM), and/or a storage device, for example, non-volatile memory, magnetic media, semiconductor memory devices, hard drive, removable storage, and optical media (e.g., DVD, CD-ROM).

Computing device 126 may include a data storage device(s) 114, which may store data, for example, ML model(s) 114A and/or one or more rule(s) 114B, for analyzing the trends of the measurements made by sensor 104-105, as described herein. Data storage device(s) 114 may be implemented as, for example, a memory, a local hard-drive, virtual storage, a removable storage unit, an optical disk, a storage device, and/or as a remote server and/or computing cloud (e.g., accessed using a network connection).

Computing device 126 may include a physical user interface 116 that includes a mechanism for user interaction, for example, to enter data and/or to view data. Exemplary physical user interfaces 116 include, for example, one or more of, a touchscreen, a display, gesture activation devices, a keyboard, a mouse, and voice activated software using speakers and microphone.

Computing device 126 may include one or more data interfaces 118 for providing communication with one or more of: temperature sensor(s) 104, other sensor(s) 105, and/or other external devices (e.g., server(s) 120 and/or client terminal(s) 122) optionally over a network 124. Data interface 118 may be implemented as, for example, one or more of, a network interface, a vehicle data interface, a USB port, a network interface card, an antenna, a wireless interface to connect to a wireless network, a short range wireless connection, a physical interface for connecting to a cable for network connectivity, a virtual interface implemented in software, network communication software providing higher layers of network connectivity, and/or other implementations.

Network 124 may be implemented as, for example, the internet, a broadcast network, a local area network, a virtual network, a wireless network, a cellular network, a local bus, a point to point link (e.g., wired), and/or combinations of the aforementioned. It is noted that a cable connecting processor(s) 110 and another device may be referred to herein as network 124.

System 100 may be implemented as different architectures. For example, in a server-client architecture, computing device 126 is implemented as a server that receives measurements of ambient temperature sensed by temperature sensor(s) 104 (and/or measurements made by other sensors 105) from a client terminal 122 over network 124, for example, a smartphone with connected temperature sensor(s) 104. Computing device 126 analyzes the temperature measurements and/or other data (e.g., physiological parameter(s) and/or location) as described herein, and may generate instructions, as described herein. The result of the analysis may be provided to the client terminal, for example, for local generation of instructions, such as an alert and/or activation of an air conditioner, as described herein. In an example of a local architecture, computing device 126 is implemented as a local computer, optionally smartphone, that receives measurements of temperature sensed by temperature sensor(s) 104 (and/or measurements made by other sensors 105) (e.g., sensors of the smartphone and/or other sensors that may be connected such as by Internet of Things (IoT) technology), locally analyzes the temperature measurements and/or other data (e.g., physiological parameter(s) and/or location) as described herein, and locally generates instructions, as described herein.

In some implementations, other sensor(s) 105 and temperature sensor(s) 104 are installed on a common object, for example, installed on a wearable garment and/or housing in a fixed position with no relative movement between the location sensor and temperature sensor 104. This enables determining the location of the temperature sensor 104 indirectly via the location sensor, as described herein. Alternatively or additionally, other sensor(s) 105 and ambient temperature sensor(s) 104 are installed at different locations. For example, ambient temperature sensor(s) 104 is installed within a smartphone, and other sensor(s) 105 (e.g., heart rate, body temperature) are installed within a garment worn by the subject. The other sensor(s) 105 may communicate with computing device 126, for example, via short range wireless connections, IoT technology, and the like.

Referring now back to FIG. 2, at 202, multiple temperature measurements are obtained by the temperature sensor, optionally by a single temperature sensor.

The multiple temperature measurements are of the ambient environment.

The multiple temperature measurements may be made continuously (or near-continuously), and/or at spaced apart intervals, for example, every about 0.01 second, or about 0.1 second, or about 0.5 second, or about 1 second, or other values. The rate of measurement may be selected according to the desired accuracy of computing the trend, for example, based on the fastest decrease or increase in temperature that is expected.

The multiple temperature measurements may be made over a time interval, which may be a sliding window, used to compute the trend. The time interval may be, for example, about 1second, or about 3 seconds, or about 5 seconds, or about 10 seconds, or about 30 seconds, or about 1 minute, or other values.

At 204, optionally, other measurements are obtained by other sensors, for example, as described herein. The other measurements may be made simultaneously with the temperature measurement, and/or near simultaneously with the temperature measurements, or at their own independent rate according to the measurement being made. The other measurement may be made within the same time interval used for the temperature measurements. The other measurements may be correlated with the temperature measurements, for example, time synchronized.

Other measurements may include location measurements obtained over the time interval by a location sensor. The location sensor is associated with the temperature sensor, such that the location measurements indicate location of the temperature sensor. For example, the location sensor and the temperature sensor are installed with a common housing, and/or fixed to each other.

Yet additional other measurements may include physiological measurements made by one or more physiological sensors. Exemplary physiological sensors and physiological measurements include: heart rate sensor for sensing a heart rate of the subject, pulse oximeter sensor for sensing oxygenation of the subject, electromyogram (EMG) sensor for sensing electrical activity of tissues, and the like.

Yet additional other measurements may include body temperature sensed by a body temperature sensor, for example, by contact and/or non-contact. It is noted that the body temperature sensor may be referred to as a physiological sensor.

Yet additional other measurements may include humidity of the ambient environment.

At 206, an analysis of location measurements obtained over the time interval by the location sensor is performed to determine how the temperature sensor was moved.

The analysis of location measurements may be performed to identify whether the temperature measurements obtained over the time interval occurred while the temperature sensor was moved independently of the subject. For example, to identify whether a device that includes the temperature sensor was taken out of a pocket of the subject and/or removed from under garments worn by the user. In such a case, the change in temperature measured by the temperature sensor is due to the local change in location of the device, for example, when the temperature sensor is in the pocket of the subject, the ambient temperature sensed is close to the body temperature of the subject. When the temperature sensor is removed from the subject, the ambient temperature sensed is the environmental temperature, for example, cold on a winter day, and hot on a summer day. In response to identifying that the temperature sensor was moved independently of the subject, the temperature measurements made over the time interval, which may include the trend, are excluded from further analysis. Since the temperature change is caused by local movement of the temperature sensor, without the subject moving to another environment, the change in temperature is irrelevant to affecting the medical condition of the subject, and/or has no impact on comfort of the subject.

The analysis to identify whether the temperature measurements obtained over the time interval occurred while the temperature sensor was moved independently of the subject may be performed, for example, after the trend indicating temperature change is identified. The analysis may be performed to determine whether the trend is significant and possibly impacting on the medical condition of the subject, i.e., due to the subject moving from an environment with one ambient temperature to another environment with another ambient temperature, or not significant and not impacting on the medical condition of the subject (i.e., local change in position independent of the subject).

Movement of the temperature measurements independently of the subject may be identified, for example, by detecting acceleration and/or change in pose of the location sensor that is correlated with a local movement of the temperature sensor from a first position on the subject (e.g., pocket, under a garment) to a second position on the subject (e.g., held in the subject's hand, to another pocket). For example, a fast acceleration and/or change in orientation of the temperature sensor may be due to the subject moving the temperature sensor from one location on the subject to another location, which is a small distance, and therefore easy to move quickly, and/or turning the temperature sensor to another orientation during the move.

Alternatively or additionally, the analysis of location measurements may be performed to identify whether the temperature measurements obtained over the time interval occurred while the temperature sensor was moved due to movement of the subject. For example, whether the subject got into a car (i.e., movement from external environment to inside a car), and/or moved from inside a warm house to outside the house (where it might be cold in the winter). In such a case, the change in temperature measured by the temperature sensor is significant, due to a real change in ambient temperature, and further analysis is done to determine possible impact on the medical condition of the subject. In response to identifying that the temperature sensor was moved as the result of movement of the subject, the temperature measurements, i.e., trend thereof, is analyzed to determine risk of the medical condition, as described herein.

Movement of the temperature measurements due to movement of the subject may be identified, for example, by identifying a change in location greater than a threshold indicating movement of the subject from a first environment to a second environment. For example, a movement of greater than about 2 meters, or 5 meters, or 10 meters, would indicate that the subject moved between different environment, such as from outside to the inside of a car, and/or from inside of a house to the street. In another example, an analysis of acceleration and/or change in pose of the location sensor that is correlated with movement of the subject between environments may be performed. For example, a relatively slow acceleration and/or non-significant change in pose of the temperature sensor may occur when the subject walk from one environment (e.g., inside a house) to another environment (e.g., outside the house) without locally moving the temperature sensor (e.g., the temperature sensor remains in the pocket, and/or held in hand, and/or worn as a necklace).

At 208, a trend of temperature measurements of the ambient temperature obtained over the time interval by the temperature sensor, is computed.

The trend may be computed over the time interval, optionally iteratively, such as by a sliding window of a size of the time interval.

The trend may include differentials between successive temperature measurements and preceding temperature measurements, for example, between maximal temperatures and minimal temperatures in the sliding window, optionally as a function of time between the maximum and minimum temperatures.

The trend may be computed by fitting a function to the temperature measurements and computing a first derivative of the function to indicate rate, and/or a second derivative of the function to indicate acceleration in change of temperature.

The trend may be a spatiotemporal trend of the temperature measurements assigned location measurement. The spatiotemporal trend may indicate for example, rate of change of temperature as a function of location. The spatiotemporal trend may be used, for example, to identify the cause of the temperature change, such as subject moved from inside a house to outside the house, and/or to indicate how to treat the subject to reduce or avoid risk of the temperature changes, for example, to move back into the house.

The trend may be, for example, an increase in temperature, a decrease in temperature, and/or instability of the temperature (e.g., rapid changes in temperature, going up and/or down).

Alternatively or additionally, one or more additional trends are computed based on additional measurements made over the time interval. For example, a trend of location measurements made by location sensor(s), a trend of physiological measurements of the subject obtained over the time interval by physiological sensor(s), a trend of body temperatures of the subject made by a body temperature sensor and/or a trend of ambient humidity.

The additional trends may include differentials between successive other measurements (e.g., body temperature, location, physiological parameter, humidity) and preceding measurements, for example, between maximal measurements and minimal measurement in the sliding window, optionally as a function of time between the maximum and minimum measurement.

The additional trends may be time synchronized with the trend of ambient measurements, and/or combined into a combination trend and/or a multi-dimensional trend where each type of measurement represents a respective dimension in a multi-dimensional space. The differentials may be between points of the multi-dimensional trend in the multi-dimensional space, for example, between a first point indicating an optimal local maximum of both ambient temperature and heart rate, and a second point indicating an optimal local minimum of both ambient temperature and heart rate.

At 210, the trend, including the differentials, may be analyzed to determine risk of triggering and/or exacerbating the medical condition of the subject. Exemplary medical conditions affected by changes in temperature, usually rapid changes in temperature, include infection with a respiratory virus (e.g., common cold, COVID), migraine, exacerbation of sinusitis, cardiovascular issues, exacerbation of arthritis, dryness of skin, worsening of asthma and/or bronchitis, overheating, and the like. The rapid changes may be, from a hot environment to a cold environment, and/or from a cold environment to a hot environment. For example, by a person stepping out from a warm home to a cold outdoor porch. In another example, by a person taking off a warm jacket when the ambient temperature is low. In yet another example, a person moving from a street during a hot summer day into a car where the air conditioner is blasting cold air.

The analysis of the trend may be done by identifying a rate of change of temperature faster than a threshold and/or other requirement such as a set of rules and/or change pattern. The change of temperature may be a decrease in temperature, and/or an increase in temperature. Rapid changes in temperature may be harder on the body, leading to increased risk of the medical condition. For example, a change of about 2 degrees Celsius occurring over 5 seconds may be significant, causing discomfort to the subject and/or risk to triggering the medical condition. In contrast, a change of about 5 degrees Celsius over a long time, such as over 30 minutes, may be insignificant, since the body is able to slowly adjust to the temperature. The threshold and/or set of rules and/or change pattern may be selected, for example, based on correlation with risk of triggering and/or exacerbating the medical condition.

Optionally, the analysis includes an analysis of a combination of the trend of ambient temperatures with the one or more other trends described herein (e.g., with reference to 208), such as trend of location, and/or trend of physiological measurements, and/or trend of body temperature, and/or trend of humidity. The analysis may be of the trend of ambient temperatures in view of the one or more other trends, such as for indicating the risk. The analysis of the combination of trends may differentiate between similar scenarios, where one scenario does not indicate a risk, while another scenario does indicate a risk. For example, in an environment with a cold ambient temperature (e.g., in winter) a subject may be exercising (e.g., walking quickly, jogging-which may be detected for example by an increased in heart rate), which may not represent a risk and/or does not represent discomfort. In contrast, in another example, a subject may be exercising (e.g., jogging, swimming) in an environment with a comfortable temperature (e.g., in an air conditioned gym and/or indoor pool), and entering a cold environment with a lower ambient temperature while stopping the exercise, which may represent a risk and/or discomfort. In another example, humidity may affect the actual impact of the temperature changes on the subject. For example, a change from a first environment at a warm temperature to a second environment at a colder temperature may indicate a risk for a first humidity, and may indicate no or a lower risk for a second humidity. In yet another example, the body temperature of the individual may indicate the actual impact of the temperature changes on the subject. For example, a change from a first environment at a warm temperature to a second environment at a colder temperature may indicate a risk when the body temperature of the subject (e.g., peripheral temperature, such as of the arms) falls and is slow to increase back to a normal range, and may indicate no risk or a lower risk when the body temperature of the subject remains stable at the normal range.

The determined risk may be, for example, a binary category (e.g., risk or no risk; uncomfortable or comfortable), a category (e.g., no risk, low risk, medium risk, high risk; very uncomfortable, somewhat uncomfortable, somewhat comfortable, very comfortable), a probability value (e.g., indicating likelihood of developing the medical condition), and the like.

The risk may be determined, for example, by applying a set of rules, a function, analysis by code, and/or feeding into a trained machine learning (ML) model. The temperature measurements and/or indication of trend, optionally in combination with one or more other types of sensor measurements and/or indication of other trends, may be fed into the ML model. The indication of the risk may be obtained as an outcome of the machine learning model.

The ML model may be trained on a multi-record training dataset. A record may include an indication of a trend of sample temperature measurements and/or the sample temperature measurements made by the temperature sensor associated with a sample individual, and optionally one or more other types of sensor measurements and/or indication of other trends. The record may include a ground truth indication of whether the sample individual developed the medical condition and/or the medical condition was exacerbated.

Exemplary architectures of the ML model include, a classifier architecture (e.g., a binary classifier, a multi-class classifier), statistical classifiers and/or other statistical models, neural networks of various architectures (e.g., convolutional, fully connected, deep, encoder-decoder, recurrent, transformer, graph), support vector machines (SVM), logistic regression, k-nearest neighbor, decision trees, boosting, random forest, a regressor, and/or any other commercial or open source package allowing regression, classification, dimensional reduction, supervised, unsupervised, semi-supervised, and/or reinforcement learning. Machine learning models may be trained using supervised approaches and/or unsupervised approaches.

At 212, instructions may be generated. The instructions may be for treatment of the subject according to the outcome of the analysis that determines risk of triggering and/or exacerbating the medical condition of the subject, for example, when the change in ambient temperature is likely to trigger and/exacerbate the medical condition such as infection with a respiratory virus (e.g., common cold, COVID). The treatment may be for reducing or eliminating the risk. The treatment may be generic, regardless of the medical condition, for avoiding or reversing the sudden change in temperature. The treatment may be specific, according to the medical condition, for reducing risk of the medical condition, for example, reducing the rate of change of temperature to a different rate depending on the medical condition.

The instructions may be for automatic treatment of the subject, for example, by sending instructions (e.g., code) for activating a device and/or automatically activating the device. Alternatively or additionally, the instructions may be for manual execution (e.g., by the subject and/or another caregiver), for example, an audio message played on speakers, and/or a text and/or image and/or video message presented on a display.

The instructions may be until a condition is met, such as that a change in the ambient temperature meets a criteria, for example, a slower rate of change (e.g., increase or decrease), and/or reversing a sudden large change in ambient temperature to a smaller change (e.g., increasing the ambient temperature from a low temperature to a higher temperature), and/or until stability in the ambient temperature is detected in subsequent time intervals (e.g., within a range that is tolerable).

Exemplary instructions, for example, for automatic execution, include:
Generating instructions for activating an air conditioner and/or air heater, for example, until subsequent trends over subsequent time intervals indicate a substantially stable temperature. For example, the temperature of the air conditioner may be increased for a cold temperature to a warmer temperature to avoid drastic drops in temperature (e.g., when entering an air conditioned building from outside on a hot day). In another example, the air heater may be activated at a slow rate to slowly increase the ambient temperature to avoid drastic increase in temperature (e.g., when entering a cold room from the outside on a cold day, the temperature of the cold room may be slowly increased).
Activating a lock on a button and/or a zipper of a wearable garment of the subject for preventing removal of the wearable garment, for example, until subsequent trends over subsequent time intervals indicate a substantially stable temperature. For example, for small children to prevent them removing a coat and/or hat until the temperature is warm enough.
Generating an alert indicating the risk, for example, an audio message played on speakers and/or presented on a display (e.g., of a smartphone). The message may warn of the risk of developing and/or exacerbating the medical condition.
Generating an audio message played over speakers and a visual presentation on a display indicating to move back to the previous environment and/or add additional clothing and/or turn on a heater or air conditioner.

Exemplary instructions, for example, for manual execution, include:
Moving the subject to a different environment with a warmer ambient temperature.
Wearing a new warm garment, or putting on a recently removed garment, for example, hat, hood, scarf, and jacket.
Activating a heater for increasing the ambient temperature.

At 214, one or more features described with reference to 202-212 are iterated, for example, over subsequent time intervals. For example, the ambient temperature in proximity to the subject is continuously monitored. In another example, the iterations may be implemented for monitoring whether the instructions for treatment were implemented, for example, when the air heater was automatically turned on, whether the change in temperature did not pose a risk. In yet another example, during the iterations, the analysis of the trend may be an analysis of the trend of current time interval in combination with historical trends of historical temperature measurements computed over preceding time intervals prior to the current time interval. The historical trends may impact the outcome of the analysis of the current time interval. For example, detecting multiple drastic and/or rapid changes in temperature over multiple preceding time intervals may greatly increase the risk of developing and/or exacerbating the medical condition in comparison to a single rapid change in temperature. For example, a person that walked out of a warm house to the street in winter, then walked back in, then walked back out, multiple times, is more likely to experienced onset and/or exacerbation of the medical condition than a person that walked out of the warm house to the street in winter a single time.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant sensors will be developed and the scope of the term sensor is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for monitoring temperature changes associated with risk of a medical condition of a subject, comprising:
at least one processor in communication with a temperature sensor configured for sensing an ambient temperature and executing a code for:
computing a trend of a plurality of temperature measurements of the ambient temperature obtained over a time interval by the temperature sensor,
wherein the trend includes differentials between successive temperature measurements and preceding temperature measurements; and
analyzing the trend including the differentials to determine risk of the medical condition of the subject.

2. The system of claim 1, further comprising code for analyzing a plurality of location measurements obtained over the time interval by a location sensor associated with the, temperature sensor, to identify whether the trend occurred while the, temperature sensor was moved independently of the subject; and
in response to identifying that the temperature sensor was moved independently of the subject, excluding the trend from the analysis to determine risk.

3. The system of claim 2, wherein computing the trend comprises computing a spatiotemporal trend of a plurality of the temperature measurements assigned the plurality of location measurement.

4. The system of claim 2, wherein identifying whether the trend occurred while the temperature sensor was moved independently of the subject comprises identifying at least one of: acceleration and/or change in pose of the location sensor, correlated with a local movement of the temperature sensor from a first position on the subject to a second position on the subject.

5. The system of claim 1, further comprising code for analyzing a plurality of location measurements obtained over the time interval by a location sensor associated with the, temperature sensor, and for analyzing the plurality of location measurements for identifying that the trend occurred while the temperature sensor was moved as a result of movement the subject, and in response to identifying that the temperature sensor was moved as the result of movement of the subject, analyzing the trend to determine risk.

6. The system of claim 5, wherein identifying that the trend occurred while the temperature sensor was moved as a result of movement the subject comprises identifying a change in location greater than a threshold indicating movement of the subject from a first environment to a second environment.

7. The system of claim 1, wherein the analyzing the trend comprises identifying a rate of the decrease faster than a threshold.

8. The system of claim 1, wherein the medical condition comprises a respiratory viral infection.

9. The system of claim 8, further comprising code for generating instructions for treating the subject for preventing the risk of the respiratory viral infection by generating instructions for reducing the risk by at least one of: moving to a different environment with a warmer ambient temperature, wearing or re-wearing warmer garments, and activating a heater for increasing the ambient temperature.

10. The system of claim 1, further comprising code for computing a second trend of a plurality of physiological measurements of the subject obtained over the time interval by at least one physiological sensor, wherein analyzing comprises analyzing the trend of the plurality of temperature measurements in combination with the second trend of the plurality of physiological measurements.

11. The system of claim 1, wherein the analyzing the trend comprises identifying a rate of increase in temperature greater than a threshold.

12. The system of claim 1, further comprising code for computing a second trend of a plurality of humidity measurements obtained over the time interval by at least one humidity sensor, wherein analyzing comprises analyzing the trend of the plurality of temperature measurements in combination with the second trend of the plurality of humidity measurements.

13. The system of claim 1, further comprising a plurality of body temperature sensors configured to sense body temperature of the subject, the plurality of body temperature sensors positioned at a plurality of spaced-apart locations, each body temperature sensor sensing a respective plurality of body temperature measurements over the time interval, wherein computing the trend further comprises computing a spatiotemporal trend of the body temperature over the time interval from the plurality of temperature sensors, and wherein analyzing further comprises analyzing the spatiotemporal trend.

14. The system of claim 1, wherein analyzing the trend comprises feeding the plurality of temperature measurements into a machine learning model, and obtaining an indication of the risk as an outcome of the machine learning model, wherein the machine learning model is trained on a multi-record training dataset, wherein a record includes a trend of a plurality of sample temperature measurements made by the temperature sensor associated with a sample individual, and a ground truth indication of whether the sample individual developed the medical condition.

15. The system of claim 1, further comprising at least one body temperature sensor configured for sensing a body temperature of the subject, the at least one body temperature sensor in communication with the at least one processor, wherein the trend comprises a first trend, and code for computing a second trend of a plurality of body temperature measurements of a body of the subject over the time interval by the at least one body temperature sensor, wherein analyzing comprise analyzing a combination of the first trend and the second trend to determine risk of the medical condition of the subject.

16. The system of claim 15, wherein the at least one body temperature sensor is installed within a wearable garment, selected from:
(i) a hat, and the at least one body temperature sensor is positioned for sensing temperature of a scalp of the subject when in use,
(ii) a scarf, and the at least one body temperature sensor is positioned for sensing temperature of air exhaled from nostrils and/or mouth of the subject when in use,
(iii) earmuffs, and the at least one body temperature sensor is positioned for sensing temperature of at least one ear of the subject when in use,
(iv) a shirt, and the at least one body temperature sensor is positioned for sensing temperature of at least one armpit of the subject when in use, and
(v) a shirt and/or pants, and the at least one body temperature sensor is positioned for sensing temperature of at least one arm and/or leg and/or a chest of the subject when in use.

17. The system of claim 1, further comprising code for generating instructions for reducing the risk, including at least one of: (i) generating instructions for activating an air conditioner and/or air heater until subsequent trends over subsequent time intervals indicate a substantially stable temperature, (ii) activating a lock on a button and/or a zipper of a wearable garment of the subject for preventing removal of the wearable garment until subsequent trends over subsequent time intervals indicate a substantially stable temperature, (iii) generating an alert indicating the risk, (iv) generating at least one of an audio message played over speakers and a visual presentation on a display indicating to move back to the previous environment and/or add additional clothing and/or turn on a heater or air conditioner.

18. The system of claim 1, wherein the time interval comprises a current time interval, and analyzing the trend comprises analyzing the trend based on the plurality of temperature measurements obtained over the current time interval in combination with historical trends of historical temperature measurements computed over preceding time intervals prior to the current time interval.

19. A computer implemented method for monitoring temperature changes associated with risk of a medical condition of a subject, comprising:
computing a trend of a plurality of temperature measurements of the ambient temperature obtained over a time interval by a temperature sensor,
wherein the trend includes differentials between successive temperature measurements and preceding temperature measurements; and
analyzing the trend including the differentials to determine risk of the medical condition of the subject.

20. A non-transitory medium storing program instructions for monitoring temperature changes associated with risk of a medical condition of a subject, which when executed by at least one processor, cause the at least one processor to:
compute a trend of a plurality of temperature measurements of the ambient temperature obtained over a time interval by a temperature sensor,
wherein the trend includes differentials between successive temperature measurements and preceding temperature measurements; and
analyze the trend including the differentials to determine risk of the medical condition of the subject.
